(19) United States
(12) Patent Application Publication
(10) Pub. No.: US 2003/0143526 A1
(43) Pub. Date: Jul. 31, 2003

(54) Recombinanr hhv8 dna

(76) Inventors: Henri-Jacques Delecluse, Munchen (DE); Manuela Kost, Munchen (DE); Wolfgang Hammerschmidt, Munchen (DE)

Correspondence Address: MCDERMOTT WILL & EMERY, 600 13TH STREET, N.W., WASHINGTON, DC, 20005-3096, US

(21) Appl. No.: 10/220,640

(22) PCT Filed: Mar. 16, 2001
(86) PCT No.: PCT/EP01/03031

(51) Int. Cl. C12Q 1/70; C07H 21/02; C07H 21/04; C12N 7/01; C12N 5/06; C12N 7/00; C12N 5/00; C12N 5/02

(52) U.S. Cl. 435/5; 435/235.1; 435/325; 536/23.72

(57) ABSTRACT

The present invention relates to recombinant HHV8 DNA as well as to methods for the preparation thereof.

The present invention relates to recombinant DNA of the HHV8 virus as well as to the preparation thereof.

A breakthrough in understanding the pathogenesis of kaposi sarcoma was the discovery of the genome of a herpes virus in kaposi sarcoma cells¹. The virus referred to as kaposi sarcoma-associated herpes virus (KSHV) or human herpes virus 8 (HHV8) encodes several cytokins (MIP, IL-6, interferon regulatory factor), cyclin homologs and G proteins acting as growth factors or stimulating cell proliferation. All these proteins may be involved in tumor growth.

Further identified have been several viral apoptosis inhibitors and viral proteins having transforming properties. For these reasons it seems very likely, that the virus is directly involved in the emergence of kaposi sarcoma and other virus-associated diseases.

However, a number of principal questions remain open:

Up to known, the target cell of HHV8 is unknown, and similarly the cellular lineage of the kaposi sarcoma is still disputed. Furthermore, it has been impossible to propagate HHV8 in culture. The virus can only be obtained if particular cell lines having a latent HHV8 infection are treated with TPA and butyrate. In addition, all data concerning the transforming potential of this virus have been derived from experiments with single viral gene products while the functions of these gene products in the context of the whole virus are still unknown.

Similarly, it is unknown whether all or only individual viral transformation products are required for cell transformation. To clarify this question it would be necessary to mutate the HHV8 viral genome in a targeted manner what would be most conveniently carried out in prokaryotic cells, for example in E. coli cells.

It is thus an object of the present invention to provide HHV8 DNA which may be replicated in prokaryotic cells such as in E. coli and which enables the production of infectious particles without using inducing agents such as TPA or butyrate.

According to the present invention, this object has been solved by providing a recombinant HHV8 DNA carrying one or more essential genes of HHV8 in an inactivated form and furthermore harbouring the information for the replication of HHV8 DNA in prokaryotic cells or yeast cells as well as one or more marker gene(s) which may be selected for in a prokaryotic organism or in a yeast cell.

The term “information for the replication” is meant to relate to such sequence regions which preferably comprise the origin of replication and optionally also binding sites for replication factors. These are responsible for the initiation of DNA replication. In their entirety they are referred to as replicons.

By introducing a prokaryotic replicon or a yeast replicon into the HHV8 DNA together with one or more marker gene(s) selectable in prokaryotic cells or in yeast cells, the replication of HHV8 DNA may be carried out in prokaryotic cells or in yeast cells and in addition it will be possible to mutagenize the HHV8 genome by methods known per se, for example to introduce deletions, insertions and particularly foreign genes into the HHV8 genome in a targeted manner.

Particularly preferred as the marker genes selectable in prokaryotic organisms or yeast cells are antibiotic resistance genes and fluorescent genes or the lacZ gene. In prokaryotic organisms, e.g. E. coli or yeast cells, such as S. cerevisiae or S. pombe, selectable antibiotic resistance genes are known to those skilled in the art. Examples are the chloramphenicol resistance gene and the ampicillin resistance gene. Examples of a gene encoding a fluorescent protein include the gfp gene. In another embodiment the present invention also comprises a marker gene which may be selected for in animal or human cells. These again include genes encoding fluorescent proteins as well as antibiotic resistance genes. Examples of antibiotic resistance genes are the hygromycin resistance gene and the neomycin resistance gene.

In a preferred embodiment of the present invention, the information for the replication in prokaryotes is derived from E. coli F factor.

Another important feature of the recombinant HHV8 DNA provided according to the present invention is the inactivation of an essential gene of HHV8. The term “essential gene” relates to genes which are essential for the regulation of viral gene expression. These include in particular viral transcription factors, i.e. the immediate-early genes, as well as the early genes of DNA replication. An example of an immediate-early gene is transfection factor ORF50. Examples of early genes of DNA replication include polymerase ORF9, the DNA-binding protein ORF6, primase ORF56, helicase ORF44, and helicase/primase ORF40/ORF41, as well as the DNA polymerase-accessory factor ORF59.

The inactivation of an essential gene may be performed for example by deletion, integration, base exchange or addition of one or more nucleotides. In a preferred embodiment of the present invention the origin of replication and/or the marker gene selectable in prokaryotes and/or eukaryotes is integrated into a gene essential for HHV8 whereby the inactivation of the essential gene as well as the integration of further features required by the present invention may be carried out in one step. It should be understood that also other embodiments are possible. Integration into the essential gene is easier and may be performed in a targeted manner if the DNA to be integrated into the essential gene is flanked by regions with homology to HHV8 which enable a homologous recombination.

Essential genes are genes which regulate virus synthesis and virus maturation.

The HHV8 genome has a size of about 170 kbp (BC3). It should be understood, however, that also other molecules of HHV8 may be prepared which have the features of claim 1 but have different sizes. Examples of these are HHV8 molecules having a size of at least 100, 120, 130, 140, 150, 170, 200, and 230 kbp. A HHV8 genome of a size of 230 kbp has formed by partial duplication of the genome. An example is the BC1 virus. Considered are also larger molecules having a size of for example 250 kbp. Due to their size, all these molecules may not be replicated and mutagenized in prokaryotes using conventional techniques.

For this reason, studies of the function of individual regions of the HHV8 genome up to now have been performed with isolated segments of HHV8 DNA and not on the whole genome.

Because of the size, a skilled artisan at first would not consider the possibility that the HHV8 DNA may be replicated in prokaryotes in the form of the whole genome. Even if he would attempt to integrate a prokaryotic replicon into HHV8 DNA this integration of the replicon would be reasonably at a site of the HHV8 genome where it would not damage essential genes in order not to impair the replication of HHV8 DNA in animal or human eukaryotic cells, the expression of HHV8 genes in these eukaryotic cells as well as the formation of viral particles. According to the present invention, it has been discovered that in this case this technique is impracticable. It has been surprisingly found that the incorporation of a prokaryotic replicon or a yeast replicon into HHV8 DNA will only be successful if replicon integration is performed within an essential gene so that this gene is inactivated.

As detailed above, it will also be successful if at least one essential viral gene is inactivated by other mechanisms for example by deletion or by base exchange. In this case integration of the replicon or yeast replicon and/or the marker genes may also be carried out into non-essential viral genes.

In a preferred embodiment of the present invention, the integration of the prokaryotic replicon or yeast replicon results in an inactivation of a gene responsible for the lytic productive phase of HHV8 , such as a protein responsible for DNA replication thereby preventing virus synthesis. Without knockout of an essential gene, for example by insertion mutagenesis, it is impossible to transfect the HHV8 genome in a stable, latent form into transformed cells, such as 293 cells.

The replicon introduced into the HHV8 genome may be any prokaryotic replicon. Examples are: P1 replicon, ColE1, SC1-10, p15A, Ti. Examples of yeast replicons are: origin of replication of “2 micron circle”, autosomal replicating sequence (ARS).

A replicon which is preferred according to the present invention is the E. coli F factor replicon. The region responsible for HHV8 DNA replication in prokaryotes at least contains the origin of replication of the F factor.

Furthermore, for the selection of the recombinant HHV8 genome in prokaryotes or yeasts marker genes are introduced into the HHV8 DNA, such as antibiotic resistance genes and fluorescent genes. Other examples are the low affinity nerve growth factor receptor INGF-R, secretory alcaline phosphatase as well as genes conferring resistance against the antibiotics puromycin, zeomycin, neomycin. Another example of an antibiotic resistance gene is chloramphenicol acetyl transferase. An example of a fluorescent gene is the “green fluorescent protein”. It is further preferred to integrate a marker gene which is selectable in animal or human eukaryotic cells into HHV8 DNA. This may again be a fluorescent protein or an antibiotic resistance gene. An example of an antibiotic resistance gene is hygromycin phosphotransferase.

Preferably, the prokaryotic replicon or yeast replicon and the marker genes are localized on a continuous region of the genome. However, they may also be introduced into HHV8 DNA in a discontinuous manner, i.e. separately. It is of importance, that at least one essential gene of HHV8 is inactivated by the integration.

Preferably, integration of foreign DNA into the HHV8 DNA is performed by homologous recombination via flanking DNA regions which flank the foreign DNA to be integrated. The flanking sequences are homologous to the sites of integration on the HHV8 DNA. The length of the flanking homologous sequences is at least 300 bp, preferably one kbp or more, such as 1, 5, 2, 2.5, and 3 kbp.

The recombination is carried out in eukaryotic cells infected by HHV8. Examples of such cell lines are cell lines BC-1, BC-2, BC-3, BCBL1, and BCP1.

These eukaryotic cells are characterized by containing HHV8 molecules which do not bear inactivated essential genes and particularly wild type HHV8 virus molecules. In these eukaryotic cells, the HHV8 genome is present for example in the latent form, i.e. no production of viruses occurs. However, also other cells may be used which are persistently infected by HHV8 as well as cells in which virus and host are in a steady state.

Successful and site-specific recombination may be detected by techniques known per se, e.g. by hybridization techniques, preferably Southern blot hybridization, as well as by PCR techniques.

The HHV8 genomes obtained in this manner should be transferable into prokaryotic organisms such as E. coli, or yeast cells. However, many subsequent experiments showed that these recombinant HHV8 genomes may not be directly introduced into E. coli. Therefore, a new strategy was developed to enable introduction of the recombinant HHV8 molecules into a prokaryotic cell or yeast cell. It has been found according to the present invention that it was successful to induce the lytic cycle in cells which were latently infected with HHV8 and contained both the recombinant whole HHV8 molecules and molecules of the HHV8 wild type genome. A possibility is the induction via chemical agents, for example phorbole esters. Incubation of these cells together with the compounds results in an induction of virus production. Since the cells bear both recombinant HHV8 genomes and wild type HHV8 genomes, a mixed population is formed, and the viruses of this mixed population are released into the cell culture supernatant.

In the next step, the recombinant virus particles are distinguished from the wild type particles. For this purpose, transformed cells, such as 293 cells, are infected with virus supernatant (which contains a mixed population of viruses). Infected cells and uninfected cells are selected by selection, for example using an antibiotic in the case the recombinant virus genomes bear an antibiotic resistance gene. An example of an antibiotic resistance gene is the hygromycin resistance gene. Thus, the transformed cells used are a kind of biological filter by which recombinant and non-recombinant wild type HHV8 viruses may be discriminated. Also in this case it was important for the success of this step that the recombinant HHV8 virus genome is present in the infected cells, such as 293 cells, in a stable, latent state. For this purpose again, and now the last piece has fallen into place, inactivation of an essential viral gene as created in the present HHV8 DNA genome is essential.

From the cells obtained in this manner, viral DNA is prepared and introduced into the prokaryotic cell, preferably an E. coli or a yeast cell. E. coli is merely a preferred prokaryotic cell used. However, also other hosts may be used, such as Enterobacteriaceae, pseudomonads, Bacilli, and also yeasts. In these cells the recombinant HHV8 genome may be altered by methods of manipulation known per se.

The procedure outlined herein for the first time led to successful cloning of the whole HHV8 genome in E. coli on the basis of a recombinant plasmid derived from an F factor. The HHV8 DNA obtained in this way may be manipulated in prokaryotes, preferably in E. coli, by methods known per se. The cloned viral genome may be introduced into transformed cells, preferably 293 cells, and in these cells infectious particles may then be produced without addition of chemical agents such as TPA or butyrate.

The transfection of recombinant HHV8-DNA may be extended to any cell which can be infected by HHV8 only with difficulty or not at all. Since the recombinant HHV8 genome provided by the present invention carries marker genes which may be identified in prokaryotes and/or eukaryotes, e.g. genes encoding fluorescent proteins such as GFP, the infection pathway of HHV8 may be directly followed, and the target cells of HHV8 may now be identified much easier as by the methods known from the prior art.

It should be understood, that the skilled artisan may carry out other modifications of the above-described method without departing from the present invention. For example the recombinant HHV8 molecules modified in E. coli may again be transfected by HHV8-infectable cells such as 293 cells. In general, indotil cells, B cells and spindle cells may be employed instead of 293 cells.

The production of viral particles is possible because the inactivated essential HHV8 gene is complemented (rescued) by methods known per se. Alternatively, the recombinant HHV8 DNA may also be introduced into mammalian cells containing wild type HHV8 DNA. Following induction of the virus production, for example by expression of early viral transcription factor genes by heterologous promoters which e.g. are introduced into the cells together with the recombinant HHV8 DNA and are e.g. constitutively expressed, the DNA plasmids may be prepared from the thus obtained viral particles and may be again transfected for example into E. coli cells. Alternatively, however, the plasmid DNA may be directly isolated from the cells infected by wild type HHV8 and recombinant HHV8 molecules and the plasmid DNA may be transfected into E. coli. In E. coli, the recombinant molecules can be distinguished from the wild type molecules by selecting for example for an antibiotic resistance gene which is only present on the recombinant molecules.

In the following, the present invention will be described with respect to the Example and the accompanying Figures to which, however, the invention is not limited. Conventional techniques known from genetic engineering are used for the practice of the present invention. In this respect see e.g. (9) and (10).

The Figures show:

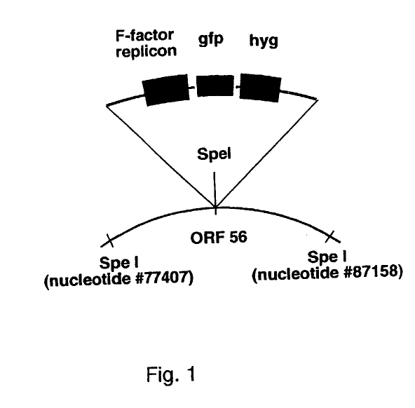

FIG. 1: A schematic representation of the construct used for homologous recombination. A plasmid backbone consisting of an F plasmid replicon, the gene for hygromycin resistance, and the GFP gene is flanked by HHV8 sequences localized to the right and to the left of the SpeI site of the ORF56 gene. By a successful homologous recombination, the F plasmid, the hygromycin resistance gene and the GFP gene are inserted into locus ORF56 while this gene is disrupted at the same time. Since cosmids of HHV8 virus strain BC1 were employed to create the flanking sequences and it is known that the sequence of this strain has several differences from virus BC3 the whole 8 kbp region surrounding the SpeI site in gene ORF56 was sequenced. Sequencing showed the presence of only four mutations.

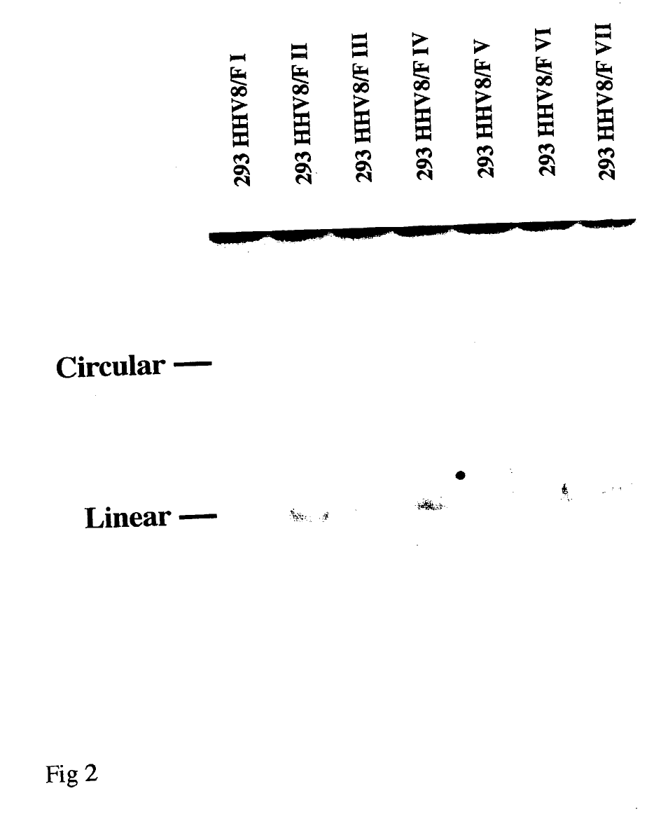

FIG. 2: Gardella gene analysis of hygromycin-resistant 293 cell clones. 293 cells were infected with supernatants of induced BC3 cell clones containing the recombinant HHV8/F plasmid. Following hygromycin selection, seven clones were analyzed with respect to the presence of circular molecules bearing the F plasmid. After hybridization with a probe specific for the F plasmid it was shown that all cell lines contained the recombinant virus genome.

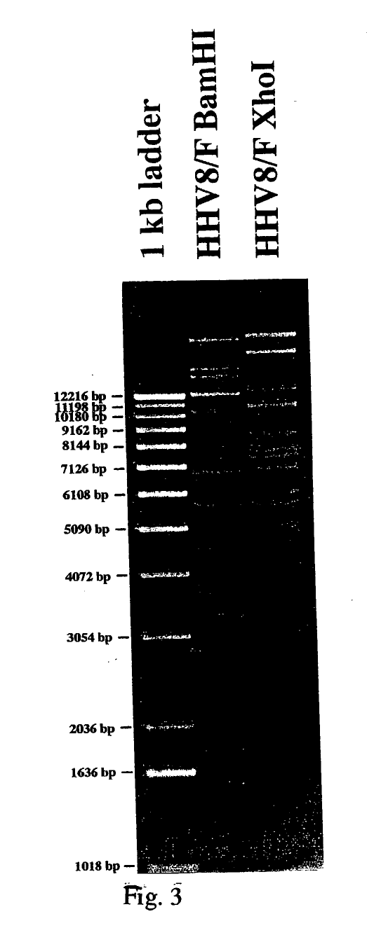

FIG. 3: Restriction analysis of recombinant HHV8 DNA. Circular molecules were extracted from a hygromycin-resistant 293 HHV/FIII cell clone supposed to contain the HHV8/F plasmid in a recombinant form. Following electroporation into E. coli strain DH10B and chloramphenicol selection the plasmid DNA was extracted and cut with restriction enzymes NheI and BamHI.

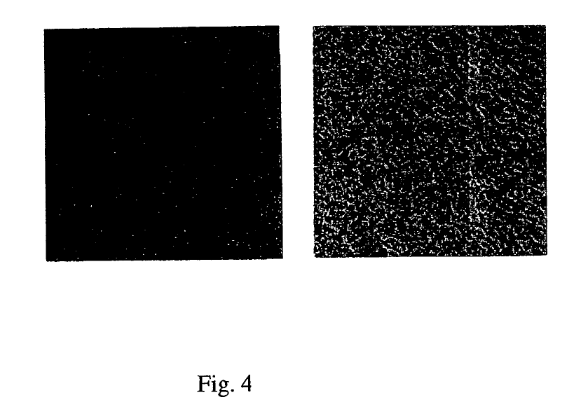

FIG. 4: Infection of 293 cells with recombinant HHV8/F plasmid virus. 293 Cell line HHV8/FIII was transfected with ORF56 cloned in an expression plasmid. After three days the supernatants of this transfected cell line were incubated with HHV8-negative 293 cells. After two days, GPF-positive, HHV8-infected 293 cells were observed.

Introduction of an F Plasmid into the HHV8 Genome

A prerequisite for the manipulation of the HHV8 genome in E. coli cells is the introduction of a prokaryotic replicon into the viral genome. Since the genome of herpes viruses is large, the F plasmid replicon was chosen which is known to accept insertions of long DNA stretches and is able to stably replicate in E. coli. The genes encoding hygromycin resistance and the green fluorescent protein were inserted providing plasmid p1919⁴. To enhance its homologous recombination with the virus genome flanking HHV8 regions were added. We decided to insert plasmid derivative 1919-F the open reading frame 56 (ORF56) of HHV8. This gene is a homolog of gene BSLF1 of the EBV virus which is required for lytic virus DNA replication. Thus, the final virus mutant is replication-incompetent while, however, the defect can be easily complemented. For this purpose we constructed the final plasmid p2421 having the p1919 backbone flanked by HHV8 sequences of about 3 and 4 kbp in length to be targeted to the ORF56 locus (FIG. 1). Subsequently, the linearized plasmid DNA fragment was introduced into cell line BC3 containing several extrachromosomal copies of the HHV8 genome. After plating into 96-well cluster plates, the cells were subjected to hygromycin selection (300 μg/ml). Four weeks following selection more than 40 hygromycin-resistant cell clones were established and characterized by means of Southern blot which were positive for the green fluorescent protein. It was found that one cell clone harboured the F plasmid correctly inserted into the HHV8 genome (data not shown). From this clone a cell culture supernatant was obtained after incubation with TPA and butyrate. This supernatant contained infectious virions, as demonstrated by expression of the green fluorescent protein following infection of 293 cells. After a selection with hygromycin (100 μg/ml) seven 293 cell clones were obtained containing the virus genome as demonstrated by Gardella gel analysis (FIG. 2). None of these 293-HHV8/F clones was able to spontaneously produce virions as had been expected by insertion mutagenesis into open reading frame 56 during the recombination. A southern blot analysis of 293-HHV8/F cell clones showed that they only contained recombinant plasmids excluding a coinfection with wild type HHV8 (data not shown).

Obtaining a Recombinant Virus Genome in E. coli

The 293-HHV8/F cell clones infected with BC3 cell line supernatant contained extrachromosomal copies of the recombinant HHV8 genome as shown in FIG. 2. Rescuing these circular molecules provided chloramphenicol-resistant E. coli cell clones obviously containing the HHV8/F hybrid plasmid (FIG. 3). A comparison of the restriction pattern obtained with that derived from an analysis of published genomic HHV8 sequences showed that the rescued genome was the complete HHV8 genome. Smaller variations with certain restriction enzymes could be demonstrated but subcloning of the HHV8/F plasmid and partial sequence determination of particular subclones confirmed the successful cloning of the BC3 genome in E. coli.

Transfection of the BSLF1 Gene Product Results in the

Production of Infectious Particles

In the next step, we wanted to examine the ability of the 293 cell line containing the recombinant HHV8/F plasmid genome to support the lytic phase of the HHV8 life cycle. Transient transfection of an expression plasmid bearing the ORF56 gene into 293-HHV8/F cells gave the production of infectious particles as indicated by an infection of the parental 293 cells (FIG. 4). The number of cells positive for the green fluorescent protein was similar to that obtained with supernatants containing 10⁵ infectious EBV particles per ml¹¹. However, it is difficult to determine the virus titer in these supernatants because it is unclear whether all 293 cell ca be infected by HHV8¹⁶. It may be concluded that with respect to infectiosness the recombinant virus is fully functional. Also 293 cells stably transfected with HHV8/F plasmid DNA purified from E. coli produced infectious virions after transfection with the ORF56 expression plasmid indicating that the passage of the virus DNA did not alter its properties.

Materials and Methods

Cells. 293 is a human embryonic kidney epithelium cell line transformed by proteins E1a and E1b of adenovirus strain 5². This cell line was cultured in RPMI 1640 supplemented with 10% fetal calf serum (Life Technologies, Eggenstein, Germany). Cell line BC3 is a body cavity lymphoma cell line shown to contain virus HHV8³. This cell line was propagated in RPMI supplemented with 20% fetal calf serum.

Recombinant DNA Plasmids

p1919 is a prokaryotic replicon based on F factor and containing the F factor origin of replication, the chloramphenicol resistance gene, the distribution genes A and B, the hygromycin resistance cassette and the gene already mentioned above encoding the green fluorescent protein. To provide flanking regions for homologous recombination with the HHV8 genome a DNA fragment (nucleotide coordinates #77407 to #87155) from the BC1 HHV8 genome⁵ derived from HHV8 cosmid GA21 was introduced into NheI restricted plasmid pACYC177 giving p2388. This subclone comprises ORF56 from HHV8 which is a homolog of gene BSLF1 of EBV. The whole plasmid p1919 was inserted into the single SpeI site of p2388 to give the final plasmid p2421 (FIG. 1).

DNA transfection. The transfection of cell lines carrying plasmid DNA was carried out using lipid micelles or electroporation. 293 cells were incubated for two hours with Optimem minimal medium (Life Technologies, Eggenstein, Germany), and the DNA embedded into lipid micelles was added for 4 hours (Lipofectamin, Life Technologies, Eggenstein, Germany). BC3 cells (10⁷ cells) were washed in RPMI 1640 without fetal calf serum, resuspended in 250 μl of the same medium and transferred together with the plasmid DNA into electroporation cuvettes having a slot width of 0.4 cm. The cells were transfected using an electroporation device (BioRad, Munich, Germany) at 230 V and 960 μF.

Hygromycin selection. One day after infection or transfection, respectively, hygromycin (Calbiochem, Germany) was added to the culture medium of BC3 or 293 cells (300 and 100 μg/ml, respectively). In intervals of one week the cells were supplied with fresh RPMI 1640 containing the same concentration of hygromycin.

Plasmid rescue in E. coli. Circular DNA molecules were isolated from F factor-positive 293 and BC3 clones using the denaturation/renaturation method as described⁶. E. coli of the strain DH10B were transformed with the isolated DNA by means of electroporation (1800 V, 25 μF, 100 Ohm). The cells were plated onto agar plates containing 15 μg/ml chloramphenicol.

Infection. 293 cells (2×10⁵) were infected with filtered (pore size 0.45 mm) supernatants of BC3/F plasmid cells in which the lytic cycle had been induced by means of TPA and butyrate, or of 293-HHV8/F plasmid cells infected with an expression plasmid containing the ORF56 gene. In some cases the 293 cells (2×10⁷) were then selected for hygromycin resistance in 6-well cluster plates and were supplied in intervals of one week with RPMI 1640 containing 10% of fetal calf serum.

Southern blot analysis and Gardella gel analysis. The method for Gardella gel electrophoresis followed by Southern blot hybridization has been already described^{7,8}.

### REFERENCES

1. Schulz, T. F. & Moore, P. S. Kaposi's sarcoma-associated herpesvirus: a new human tumor virus, but how? Trends Microbiol 7, 196-200 (1999).

2. Graham, F. L., Smiley, I., Russell, W. C. & Nairn, R. Characteristics of a human cell line transformed by DNA from human adenovirus type 5. J. Gen. Virol. 36, 59-74 (1977).

3. Arvanitakis, L., et al. Establishment and characterization of a primary efflision body cavity-based) lymphoma cell line (BC-3) harboring kaposi's sarcoma-associated herpesvirus (KSHV/HHV-8) in the absence of Epstein-Barr virus. Blood 88, 2648-54 (1996).

4. Delecluse, H. J., Hilsendegen, T., Pich, D., Zeidler, R. & Hammerschmidt, W. Propagation and recovery of intact, infectious Epstein-Barr virus from prokaryotic to human cells. Proc. Natl. Acad. Sci. USA 95, 8245-50 (1998).

5. Russo, J. J., et al. Nucleotide sequence of the Kaposi sarcoma-associated herpesvirus (HHV8). Proc. Natl. Acad. Sci. USA 93, 14862-7 (1996).

6. Griffin, B. E., Björk, E., Bjursell, G. & Lindahl, T. Sequence complexity of circular Epstein-Barr virus DNA in transformed cells. J. Virol. 40, 11-19 (1981).

7. Delecluse, H. -J., Schuller, S. & Hammerschmidt, W. Latent Marek's disease virus can be activated from its chromosomally integrated state in herpesvirus-transformed cells. EMBO J. 12, 3277-3286 (1993).

8. Gardella, T., Medveczky, P., Sairenji, T. & Mulder, C. Detection of circular and linear herpesvirus DNA molecules in mammalian cells by gel electrophoresis. J. Virol. 50, 248-254 (1984).

9. Ibelgaufts, H., Gentechnologie von A -Z, VCH, Weinheim 1990 und nachfolgende Auflagen.

10. Maniatis, Molecular Cloning, A Laboratory Manual, 1982, CSHL Press und nachfolgende Auflagen.

1. A recombinant HHV8 DNA wherein said DNA contains at least the information for the replication of HHV8 DNA in prokaryotes or yeasts as well as one or more maker gene(s) selectable in prokaryotes or yeasts and wherein at least one essential gene of HHV8 has been inactivated.

2. A HHV8 DNA according to claim 1 wherein said DNA further contains a marker gene which is selectable in animal or human eukaryotic cells.

3. A HHV8 DNA according to claim 1 or 2 wherein said marker gene is an antibiotic resistance gene or a gene encoding a fluorescent protein or the lacZ gene.

4. A HHV8 DNA according to one or more of the preceding claims wherein said essential gene has been inactivated by integration, deletion, addition or exchange of one or more nucleotides.

5. A HHV8 DNA according to one or more of the preceding claims wherein said essential gene is a gene encoding regulation of gene expression or encoding viral transcription factors.

6. A HHV8 DNA according to one or more of the preceding claims wherein the inactivation in the essential gene occurs in a gene encoding a protein for HHV8 DNA replication.

7. A HHV8 DNA according to one or more of the preceding claims wherein at least the information for replication in prokaryotes is derived from the E. coli F factor plasmid.

8. A HHV8 DNA according to one or more of the preceding claims wherein said DNA has a size of at least 100, 150, 170, 200, or up to 230 kbp or more.

9. A prokaryotic or a yeast cell containing the HHV8 DNA according to one or more of the preceding claims.

10. A prokaryotic cell according to claim 9 which is an E. coli cell.

11. An animal or human eukaryotic cell containing a recombinant HHV8 DNA according to one or more of the preceding claims.

12. A eukaryotic cell according to claim 11 further containing the genetic information encoding the inactivated essential gene.

13. A eukaryotic cell according to one or more of the preceding claims wherein said cell further contains HHV8 DNA in which essential genes can not be inactivated.

14. A eukaryotic cell according to one or more of the preceding claims wherein the HHV8 DNA with essential genes which can not be inactivated is present in a latent form.

15. HHV8 virus particles containing HHV8 DNA according to one or more of the preceding claims.

16. A method for the preparation of a recombinant HHV8 DNA wherein said method comprises the introduction of DNA into the HHV8 genome which enables replication of the HHV8 DNA in prokaryotes or yeasts, and of one or more marker gene(s) selectable in prokaryotes or yeasts, as well as the inactivation of at least one gene essential for HHV8.

17. A method according to claim 16 wherein said DNA introduced is flanked by regions of the HHV8 genome enabling a homologous recombination with HHV8 DNA.

18. A method according to one or more of the preceding claims wherein said flanking homologous HHV8 sequences have a size of ≧300 bp, preferably ≧1 kbp or more.

19. A method according to one or more of the preceding claims wherein said DNA sequences introduced are derived from the E. coli F plasmid.

20. A method according to one or more of the preceding claims wherein said homologous flanking sequences are derived from a gene essential for HHV8.

21. A method according to one or more of the preceding claims wherein the homologous flanking sequences are derived from a gene responsible for lytic viral DNA replication of HHV8.

22. A method according to one or more of the preceding claims wherein said recombinant HHV8 DNA containing at least one essential gene of HHV8 which has been inactivated, a marker gene selectable in prokaryotic cells or in yeast cells as well as the information for the replication of HHV8 DNA in prokaryotes or yeast cells is introduced into an animal or human eukaryotic cell which contains HHV8 DNA without inactivation of the essential gene.

23. A method according to claim 22 wherein said HHV8 DNA is present in a linearized form.

24. A method according to one or more of the preceding claims wherein in said eukaryotic cells containing the recombinant HHV8 DNA and the HHV8 DNA without inactivation of the essential gene the HHV8 lytic cycle is induced in order to induce the production of recombinant HHV8 virus genome.

25. A method according to claim 24 wherein a selection is performed for such eukaryotic cells containing recombinant HHV8 DNA.

26. An RNA derived from the HHV8 DNA according to one or more of claims 1 to 8.

27. The use of a recombinant DNA or RNA according to one or more of the preceding claims for further mutagenesis of HHV8 genes and for functional studies of HHV8.
